(19) United States
(12) Patent Application Publication
(10) Pub. No.: US 2012/0035473 A1
(43) Pub. Date: Feb. 9, 2012

(54) LAPAROSCOPIC HIFU PROBE

(75) Inventors: Narendra T. Sanghvi, Indianapolis, IN (US); Ralf Seip, Indianapolis, IN (US); Russell J. Fedewa, Indianapolis, IN (US); Roy F. Carlson, New Palestine, IN (US); Wo-Hsing Chen, Fishers, IN (US); Artur P. Katny, Ingalls, IN (US)

(73) Assignee: FOCUS SURGERY, INC., Indianapolis, IN (US)

(21) Appl. No.: 12/981,076

(22) Filed: Dec. 29, 2010

Related U.S. Application Data
(63) Continuation of application No. 11/444,986, filed on Jun. 1, 2006, now abandoned.
Application No. 11/444,986 is a continuation-in-part of application No. 10/380,031, filed on Mar. 10, 2003, now abandoned.
(60) Provisional application No. 60/686,499, filed on Jun. 1, 2005.

(51) Int. Cl. A61N 7/00; A61B 8/14

(52) U.S. Cl. 600/439

(57) ABSTRACT

A high-intensity focused ultrasound ablation of tissue using minimally invasive medical procedures is provided.

### CROSS REFERENCE TO RELATED APPLICATIONS

This application is a continuation-in-part of U.S. application Ser. No. 10/380,031, filed on Sep. 19, 2001, which is expressly incorporated by reference herein. This application also claims the benefit of U.S. Provisional Application Ser. No. 60/686,499, filed on Jun. 1, 2005, which is also expressly incorporated by reference herein.

### BACKGROUND AND SUMMARY OF THE INVENTION

The present invention relates to instruments to conduct minimally invasive medical procedures with the aid of laparoscopic techniques, and to such procedures themselves. More particularly, the present invention relates to high-intensity focused ultrasound ablation of tissue using minimally invasive medical procedures. It is disclosed in the context of high-intensity focused ultrasound ablation of kidney tissue, but is believed to be useful in other applications as well.

Several minimally invasive and non-invasive techniques for the treatment of living tissues and organs with ultrasound, including high-intensity, focused ultrasound, sometimes referred to hereinafter as HIFU, are known. There are, for example, the techniques and apparatus described in U.S. Pat. Nos. 4,084,582; 4,207,901; 4,223,560; 4,227,417; 4,248,090; 4,257,271; 4,317,370; 4,325,381; 4,586,512; 4,620,546; 4,658,828; 4,664,121; 4,858,613; 4,951,653; 4,955,365; 5,036,855; 5,054,470; 5,080,102; 5,117,832; 5,149,319; 5,215,680; 5,219,401; 5,247,935; 5,295,484; 5,316,000; 5,391,197; 5,409,006; 5,443,069; 5,470,350; 5,492,126; 5,573,497; 5,601,526; 5,620,479; 5,630,837; 5,643,179; 5,676,692; 5,840,031. The disclosures of these references are hereby incorporated herein by reference.

HIFU Systems for the treatment of diseased tissue are known. An exemplary HIFU system is the Sonablate® 500 HIFU system available from Focus Surgery located at 3940 Pendleton Way, Indianapolis, Ind. 46226. The Sonablate® 500 HIFU system uses a dual-element, confocal ultrasound transducer which is moved by mechanical methods, such as motors, under the control of a controller. Typically one element of the transducer is used for imaging and the other element of the transducer is used for providing HIFU Therapy.

The Sonablate® 500 HIFU system is particularly designed to provide HIFU Therapy to the prostate. However, as stated in U.S. Pat. No. 5,762,066, the disclosure of which is expressly incorporated by reference herein, the Sonablate® 500 HIFU system and/or its predecessors may be configured to treat additional types of tissue.

Further details of suitable HIFU systems may be found in U.S. Pat. No. 5,762,066; U.S. Abandoned patent application Ser. No. 07/840,502 filed Feb. 21, 1992, Australian Patent No. 5,732,801; Canadian Patent No. 1,332,441; Canadian Patent No. 2,250,081; and U.S. Pat. No. 6,685,640, the disclosures of which are expressly incorporated by reference herein.

As used herein the term “HIFU Therapy” is defined as the provision of high intensity focused ultrasound to a portion of tissue. It should be understood that the transducer may have multiple foci and that HIFU Therapy is not limited to a single focus transducer, a single transducer type, or a single ultrasound frequency. As used herein the term “HIFU Treatment” is defined as the collection of one or more HIFU Therapies. A HIFU Treatment may be all of the HIFU Therapies administered or to be administered, or it may be a subset of the HIFU Therapies administered or to be administered. As used herein the term “HIFU System” is defined as a system that is at least capable of providing a HIFU Therapy.

According to an aspect of the invention, an apparatus and method employ first, second and third devices for introduction of equipment into, and removal of equipment from, a body region, an optical imaging system, a source of a relatively non-reactive fluid for expanding the body region to facilitate the introduction of components of the apparatus into the body region and manipulation of the introduced components of apparatus, and an ultrasound apparatus for at least one of visualization and treatment of the body region. A first of the devices facilitates passing of the component of the optical imaging system into and out of the body region. A second of the devices facilitates passing the fluid between the fluid source and the body region. A third of the devices facilitates passing the ultrasound visualization and/or treatment apparatus into and out of the body region.

The laparoscopic probe of the present invention is targeted for minimally invasive laparoscopic tissue treatments. However, the probe may also be used for non-laparoscopic procedures as discussed below. The probe is light weight, easy to use, and adaptable to the current Sonablate® 500 HIFU system. The laparoscopic probe, with the Sonablate® 500 system, illustratively provides laparoscopic ultrasound imaging, treatment planning, treatment and monitoring in a single probe. The probe fits through a trocar (illustratively an 18 millimeter diameter trocar). A coupling bolus covers the tip of the probe. The bolus is very thin and illustratively expands to about five or six times its size when water is introduced. This provides a water medium surrounding the probe which is needed for ultrasonic imaging and treatment. The probe is USP Class VI certified. Cooling the transducer that provides the imaging and treatment is achieved through a sterile, distilled, degassed passive recirculating water system. The entire probe is ethylene oxide (EO) sterilizable, and the cooling system is gamma-sterilizable. Therefore every component of the probe is able to withstand repeated EO sterilization.

The laparoscopic probe of the present invention provides an alternative solution to invasive surgery. As a result, recovery time is reduced and hospital visits are considerably shorter. In addition the ablation provided by the laparoscopic probe permits the surgeon to target tissue without stopping the blood supply to the organ. For example, to perform a partial nephrectomy in a conventional manner, the surgeon illustratively shuts off the supply of blood to the kidney and has a limited amount of time to excise the targeted tissue, seal the blood vessels and restart the blood supply to the kidney. If the surgeon takes too long, damage to the kidney and possible organ death may occur. Thus being able to treat large and small volumes of tissue while permitting blood flow to the organ is a significant contribution.

Additional features of the present invention will become apparent to those skilled in the art upon consideration of the following detailed description of illustrative embodiments exemplifying the best mode of carrying out the invention as presently perceived.

### BRIEF DESCRIPTION OF THE DRAWINGS

The invention may best be understood by referring to the following detailed description and accompanying drawings which illustrate the invention. In the drawings:

FIG. 1 illustrates a partly block diagrammatic, partly fragmentary perspective view of a procedure according to the present invention;

FIG. 2 illustrates an exploded, fragmentary perspective view of a device useful in the conduct of the procedure illustrated in FIG. 1;

FIG. 3 illustrates a perspective view of another device constructed according to the invention;

FIG. 4 illustrates a perspective view of another device constructed according to the invention;

FIG. 5 illustrates a perspective view of certain components of another device constructed according to the invention;

FIG. 6 illustrates a plan view of the components illustrated in FIG. 5;

FIG. 7 illustrates an elevational view of the components illustrated in FIGS. 5-6;

FIG. 8 illustrates an end elevational view of the components illustrated in FIGS. 5-7;

FIG. 9 is a perspective view of a portion of a laparoscopic probe of another illustrated embodiment of the present invention including a controller, a drive mechanism, and a movable transducer;

FIG. 10 is a perspective view of a probe tip assembly of another illustrated embodiment of the present invention including an expandable bolus for acoustically coupling the transducer to a targeted area and for cooling the transducer during the procedure;

FIG. 11 is an exploded perspective view of the probe tip assembly of FIG. 10;

FIG. 12 is a side elevational view of the probe tip assembly of FIGS. 10 and 11;

FIG. 13 is a sample screen shot for planning a HIFU Treatment;

FIGS. 14A-14C illustrate a treatment path along which the transducer is moved by the controller and drive mechanisms to treat a treatment zone; and

FIG. 15 is a screen shot illustrating a sample procedure in accordance with an illustrated embodiment of the present invention.

### DETAILED DESCRIPTIONS OF ILLUSTRATIVE EMBODIMENTS

Although the illustrated embodiment is shown in connection with treatment of a kidney 40, the illustrated probe, water circulation system and treatment is not limited to kidneys. The present invention is presently believed to be applicable equally readily to the ablation of tissue of the liver, the pancreas, the urinary bladder 32, the gall bladder, the stomach, the heart, lungs, uterus or any other organ suitable for treatment by HIFU Therapy. In addition, the probe assembly and other features of the present invention described herein may be used in conventional non-laparoscopic HIFU Therapy of, for instance, the prostrate, esophagus, vagina, or the like.

In an illustrated minimally invasive, HIFU-based procedure, the patient 20 is first prepared by the insertion of a guide wire 24 through the urethra 28 and bladder 32 into the ureter 36 of a diseased kidney 40. The guide wire 24 is, of course, radiopaque, so that its progress to the surgical field can be straightforwardly monitored. Then, using the guide wire 24, a urological catheter 44 is inserted along the same path to permit the introduction of fluid species into the surgical site 48. Next, three incisions 50, 52, 54 are made on the abdomen 56 below the diaphragm through trocars 60. The trocars 60 are left in place, as is customary, to permit the sealing of the abdomen 56 when instruments are passed through the seals 64 of the trocars 60 into the abdomen 56 for the conduct of the procedure.

A laparoscope 68 for providing visual observation of the surgical field is passed through one of the trocars 60. The laparoscope 68 is conventionally coupled to a video camera 72 and a light source 76 for illuminating the surgical field and returning images to a surgical monitor 84. The laparoscope provides a pair of fiberoptic ports, one an output port for light from source 76 to the surgical field, and one an input port for the returning image information to video camera 72. A second of the trocars 60 provides, among other things, a passageway for the introduction into the abdomen 56 of a relatively inert gas, such as, for example, carbon dioxide, from a source 88 in order to permit the inflation of the abdomen 56 below the diaphragm. This increases the space inside the abdomen 56 for maneuvering surgical instruments including the laparoscope 68, and provides a clearer view of the surgical field.

The third trocar 60 provides access through the abdominal wall and into the surgical field for a HIFU probe 90 which will be used to ablate the surgical site 48 of a diseased kidney 40, for example, for the virtually bloodless ablation of (a) tumor(s) on the surface of, and/or within, the kidney 40. Should the surgical procedure call for it, additional trocars 60 can, of course, be provided for passing into the body additional HIFU probes 90 to be used in conjunction with each other in an ablation procedure. The presence of the catheter 44 in the kidney 40 also permits the introduction into the surgical field of (an) ablation enhancing medium (media) and other media at (an) appropriate time(s) during the procedure. The same, or a different, medium (media) may also be introduced through the catheter 44 to improve the accuracy of the targeting of the surgical site 48 for ablation and provide feedback to the treating physician of the progress of the treatment. For example, lesions which are not on the surface of the tissue 40 being treated are not easily visible, or in many cases visible at all, in the laparoscopically informed monitor 84.

In order to provide feedback to the treating physician of the progress of treatment of a site 48 not visible on the monitor 84, the ultrasound probe 90 includes an ultrasound visualization capability. (An) additional mechanism(s) may be provided for essentially real-time monitoring of the progress of the treatment. For example, it is known in the ultrasound visualization and therapy arts that there are numerous mechanisms available to promote visualization of the progress of ultrasound treatment within an organ or tissue. These include the introduction of relatively inert gas-containing microcapsule- or microbubble-seeded species, such as sterile saline solution, the introduction of a relatively inert gas, again, such as carbon dioxide, and so on. Any suitable one or ones of these mechanisms can be used to introduce any of such media via the catheter 44 into the kidney 40 being treated. Such materials are known to create bright echogenic bands, strips, fields, and the like on, for example, B-mode ultrasound imaging scans 86. Such phenomena can be used to indicate to the treating physician where the HIFU has been effective. The treating physician continues to expose the tissue 40 under treatment to the HIFU until the material produces a “bloom” or bright echogenic field (“popcorn”), band, strip or the like in the ultrasound image 86 of the treatment field. Then the HIFU probe 90 is repositioned to treat the next region which is to be treated according to the treatment regimen. Some of such species, such as relatively inert gas-containing microcapsule-seeded sterile saline solution, microbubble-seeded sterile saline solution, and the like, may also function to enhance the ablation effects of the applied HIFU. For example, some of such species readily produce cavitation, the bursting of bubbles created when the species are exposed to HIFU above certain field strengths and/or for certain lengths of time. The cavitation is known to cause further mechanical alteration of the character of the tissue at the surgical site 48 at a cellular level, enhancing the effects of the HIFU exposure. This ultimately results in reduced treatment times.

As discussed above, this treatment is not limited to kidneys. It is presently believed to be applicable equally readily to the ablation of tissue on the surface of, or in the bulk of, for example, the liver, the pancreas, the urinary bladder 32, the gall bladder, the stomach, the heart, lungs, and so on.

Turning now to the construction of the HIFU probe 90 and related hardware, although the probe 90 was tested by manipulation by the treating physician, it is within the contemplation of the present invention that the probe 90 could be integrated into, or mounted to be manipulated by, a robotic mechanism 92, and controlled, for example, by means of a joystick 94, keypad 96, programmable machine 100, or any other appropriate control mechanism. Any of such mechanisms 92, 94, 96, 100 can incorporate feedback control (illustrated by broken lines), not only of a visual nature, provided via a laparoscope 68, but also of the ultrasound imaging type via probe 90.

The ultrasound image 86 feedback may be not only of the more conventional type described above, but also, may be of a somewhat more highly processed nature, such as that described in, for example, PCT International Pub. No. WO 01/82777, titled Non-Invasive Tissue Characterization, assigned to the assignee of this application, and hereby incorporated herein by reference. It is contemplated that the feedback could provide the treating physician with highly detailed information on the progress of treatment, such as, for example, when the tissue being treated reaches a particular temperature, when the character of the tissue at a cellular level changes abruptly, and so on.

The illustrated probe 90 itself is, for example, a modified Sonablate 200 probe available from Focus Surgery, Inc., 3940 Pendleton Way, Indianapolis, Ind., 46226. The Sonablate 200 system is hereby incorporated herein by reference. The probe 90 includes a segmented, curved rectangular elliptical transducer 104 of the general type described in, for example, WO 99/49788. The transducer 104 has a central segment 108 which is used both for visualization and therapy and (an) outer segment(s) 112 which is (are) used for therapy, in accordance with known principles. However, it will immediately be appreciated that other single element or multi-segment transducer configurations, such as ones providing variable focal length, can be used to advantage in other embodiments of the invention. Some of such variable focal length configurations, and driving and receiving systems for them, are described in the prior art incorporated herein by reference.

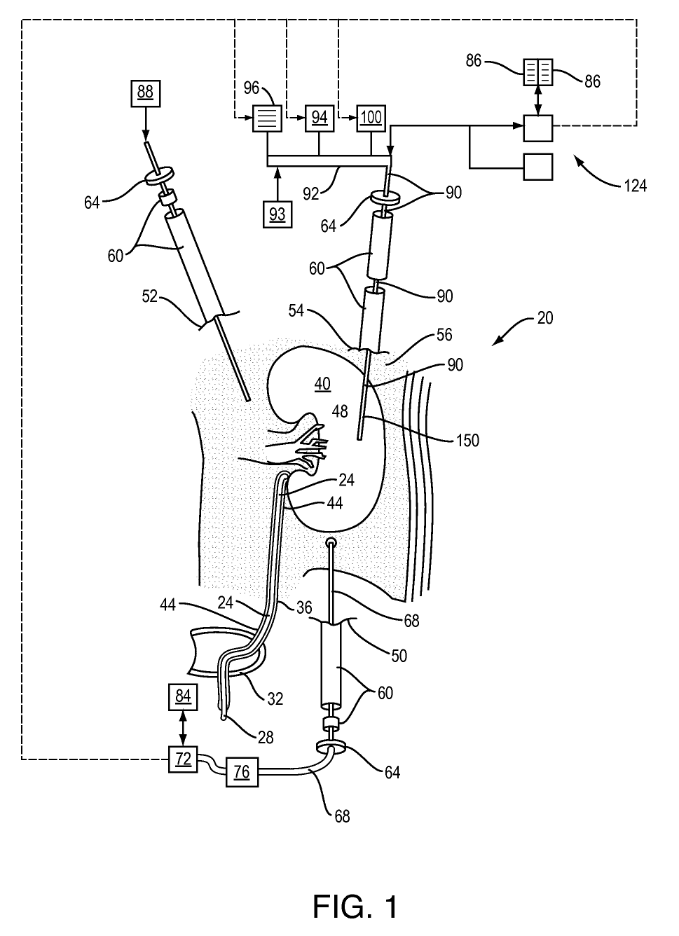

The illustrated transducer 104 has a length of about 3 cm., a width of about 1.3 cm., and a focal length of about 3.5 cm. This is adequate to treat tumors of the kidney 40 to that depth. The HIFU treatment of deeper seated tissue will, of course, require longer focal length treatment transducers. The transducer 104 is mounted in a holder 116 having the same generally rectangular prism-shaped outline as the outer dimensions of the transducer 104 itself. The holder 116 is mounted on the end of a hollow shaft 120 through which the electrical leads to drive the transducer 104 for imaging 86 and therapy can be passed between the transducer 104 and the driver and imaging circuitry, for example, the driver and imaging circuitry of the above-mentioned Sonablate 200 system, in a controller 124 (FIG. 1). The shaft 120 itself can serve as one of the conductors, for example, the ground conductor, for one or more of the ultrasound-generating segment(s) 108, 112 of the transducer 104. The transducer 104/holder 116/shaft 120 assembly is housed in a housing 128 which illustratively is about 50 cm in length and has an outside diameter which is sufficiently small to fit through one of the standard trocar 60 seals 64, for example, an 18 mm seal 64, sufficiently tightly to seal the inside of the abdominal cavity in use. Of course, the dimensions of the illustrated transducer 104, holder 116 and housing 128 given above are for a probe 90 for the treatment of certain kidney 40 tissue. The size, shape and focal length of the probe 90 and transducer 104 will depend to a great extent on the requirements of the tissue or organ which the probe 90 is intended to treat. For example, a liver probe may be required to be somewhat larger and have a longer focal length, and so on.

It should be recalled that it is contemplated that the abdominal cavity will be pressurized with gas during the procedure to increase the work space inside the abdominal cavity. Recalling that a gas will ordinarily be used during the procedure to inflate the abdomen 56, provision must be made for coupling the ultrasound transducer 104 to the tissue being treated. This may be done by providing a cot or condom 132 over the window 136 through the housing 128 through which the ultrasound radiating face 140 of the transducer 104 transmits ultrasound, and filling the housing 128 with an appropriate coupling medium, for example, degassed and sterile water and permitting air to escape from the housing 128 as it is being filled. One or more ports may be provided in the housing 128 for filling it with coupling medium and bleeding air from it. The cot 132 may be sealed to the housing 128 longitudinally of the housing 128 on either side of the window 136 by elastomeric O-ring seals 144. This reduces the amount of coupling fluid necessary inside the housing 128 to cause the cot 132 to bulge out sufficiently to bring it into intimate contact with the surface of the tissue 40 to be treated.

To reduce further the amount of coupling fluid necessary inside the housing 128 to cause the cot 132 to bulge out sufficiently to bring it into intimate contact with the surface of the tissue 40 to be treated, a sleeve 148 having an opening 152 corresponding generally in size, shape and orientation to the size, shape and orientation of the window 136, such as, for example, a longitudinally slitted 152 sleeve 148, is placed around the housing 128 in the region of the window 136. The sleeve 148 illustratively is constructed of a thin, sterilizable or sterile disposable material, such as, for example, a resin or light metal. The sleeve 148 slides or snaps around the housing 128 in the region of the ultrasound window 136 after the cot 132 has been placed over the window 136, and either before or after the O-rings 144 have been positioned adjacent the longitudinal ends of the window 136. The sleeve 148 is intended to reduce the bulging of the cot 132 anywhere other than in the immediate vicinity of the window 136. This reduces the amount of coupling fluid necessary to cause the cot 132 to bulge into intimate contact with the tissue 40 by reducing the volume of coupling fluid necessary to cause adequate bulging of the cot 132.

It should also be recalled that ultrasound tissue imaging 86 is deep tissue imaging, not surface imaging. Surface imaging in the illustrated application is provided by the laparoscope 68's vision system 76, 72, 84. It is helpful for both gross and fine positioning of the probe 90, including tissue contact with the cot 132 filled with coupling medium, and for monitoring the progress of treatment. For example, visualization permits the physician to determine when the tissue 40 being treated exhibits surface blanching 156 (FIG. 1). The presence of blanching 156 provides visual feedback to the treating physician that the tissue 40 being treated has received an amount of heat, at least on its surface, to achieve a particular level of ablation. Instead of this surface imaging being provided laparoscopically, this surface imaging could also be provided by means of a light source and video return on the probe 90 itself. The light source and video return on the probe 90 itself might take the form of an LED or other light source provided on the probe 90 adjacent the window 136, and a miniature video image generator of some type also adjacent the window 136, or some other combination of image-generating components.

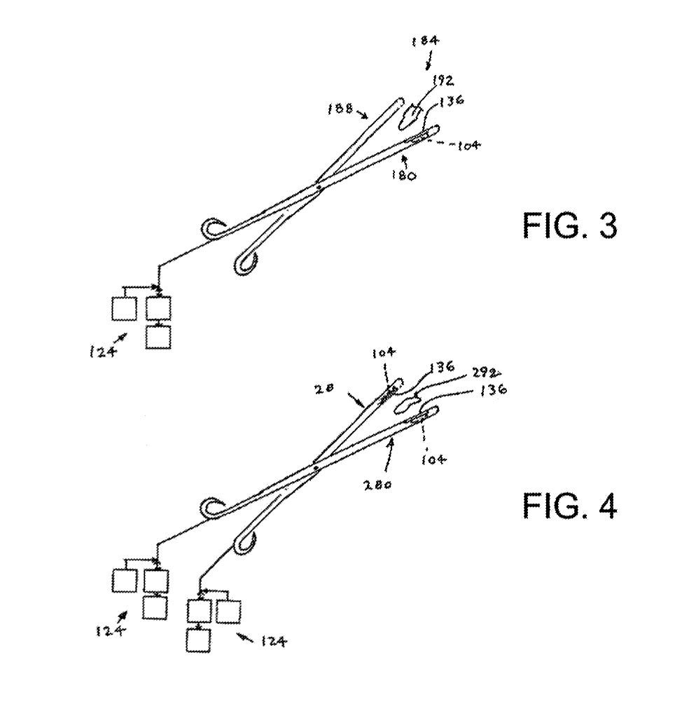

In another embodiment, illustrated in FIG. 3, the probe 180 takes the form of one jaw of a forceps-like clamp 184. The other jaw 188 of the clamp 184 serves with the clamping jaw/probe 180 to capture the tissue 192 to be treated between the two jaws 180, 188. Then, the transducer 104 in the jaw 180 is energized in the same way as discussed above by a driver/receiver/visualization system 124 to treat the tissue 192 with HIFU. In another embodiment, illustrated in FIG. 4, both jaws 280, 288 can take the form of probes so that the tissue 292 to be treated could be treated by both probes 280, 288 or by whichever one of the probes 280, 288 is optimally positioned to treat the tissue 292 to be treated. The ultrasound transducers 104, 104 in the two probe/jaws 280, 288 could have different characteristics, for example, different power handling capabilities or focal lengths, in order to provide a greater number of treatment options to the physician when the probes/jaws 280, 288 are in position to treat the tissue 292.

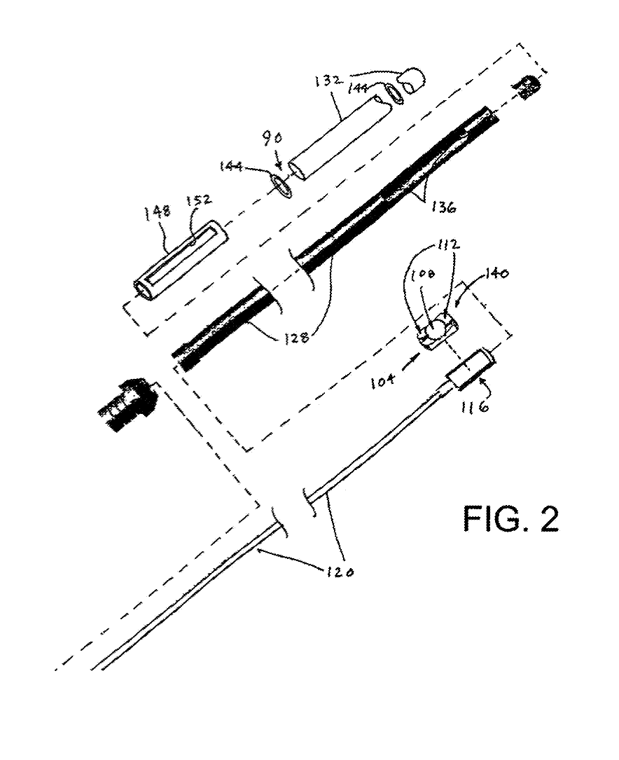

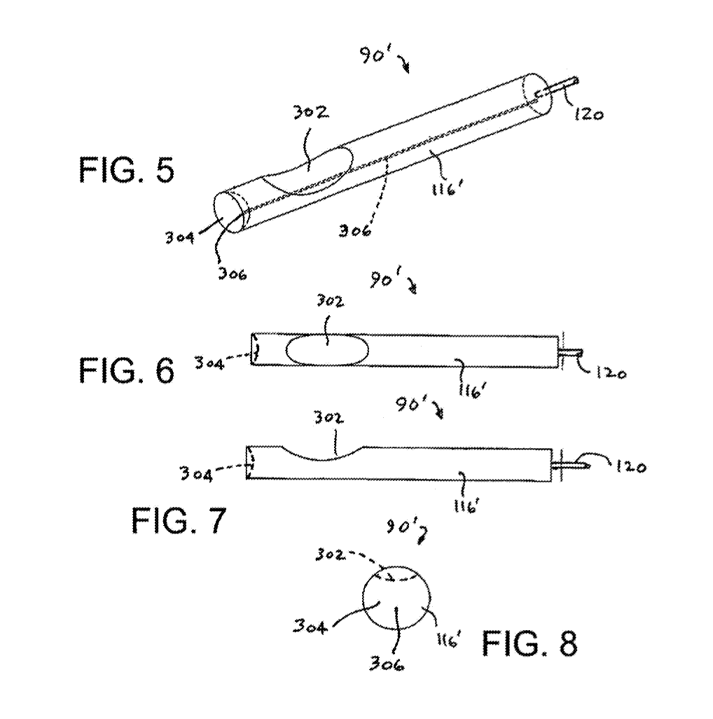

In another embodiment, illustrated in FIGS. 5-8, a probe 90′ includes a holder 116′ for mounting part-spherical visualization and treatment transducers 302, 304 having radii of, for example, 30 mm for transducer 302 and 15 mm for transducer 304. Both of transducers 302, 304 are capable of operation in visualization and HIFU treatment modes. And, of course, either or both of transducers 302, 304 can be a multi-element transducer of any of the known types including transducer 104 illustrated in FIGS. 1-2. In this embodiment, the end cap and the end O-ring seal 144 of the embodiment illustrated in FIGS. 1-2 are omitted to permit the cot 132 to bulge from the end of probe 90′ when the cot 132 is filled with coupling medium, in order that ultrasound may better be coupled from/to the transducer 304 to/from tissue being visualized and/or treated. Holder 116′ also includes its own fiberoptic passageway 306 having a diameter of, for example, 0.5 mm. Passageway 306 extends out to the surface of transducer 304 to provide optical visualization of tissue being treated, which tissue may also be visualized by ultrasound and/or treated by transducer 304. The optical fiber(s) which extend(s) through passageway 306 is (are) coupled to an illumination/optical visualization system of known type, such as the system 72, 76, 84 illustrated and briefly described in connection with the embodiment illustrated in FIGS. 1-2.

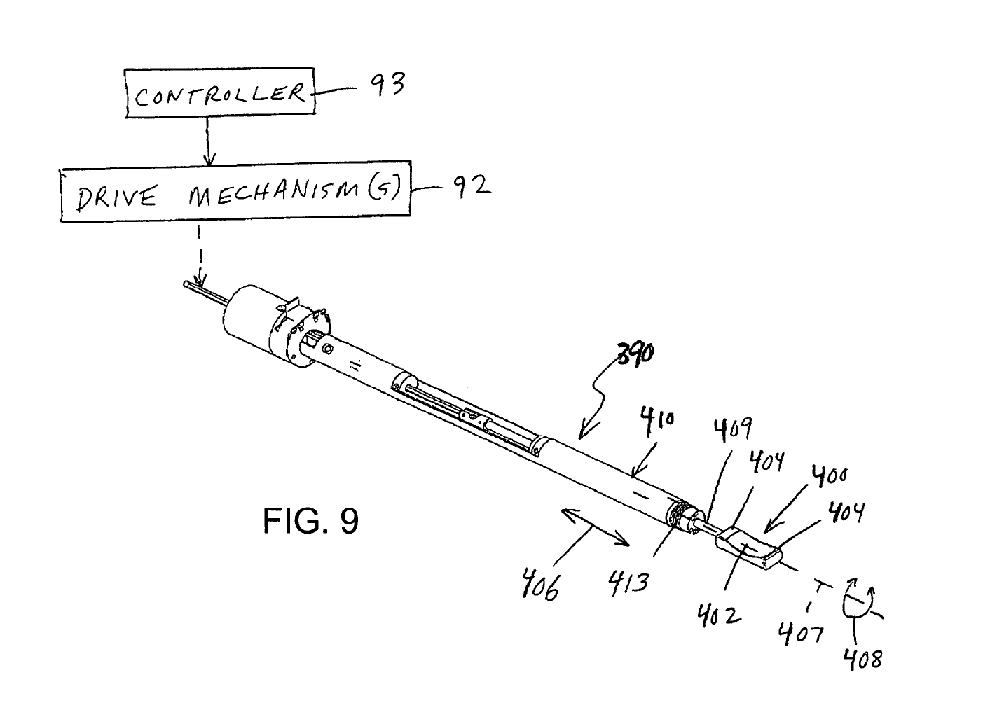

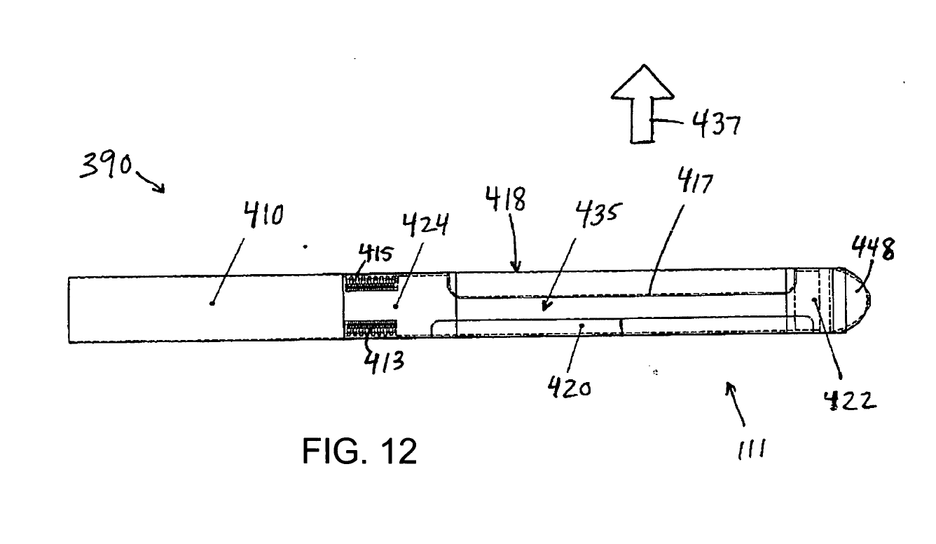

Turning now to the construction of another embodiment of the HIFU probe and related hardware shown in FIGS. 9-12, the probe 390 is illustratively integrated into, or mounted to be manipulated by, a drive mechanism 92, and controlled, for example, by means of a joystick 94, keypad 96, touch screen 100, or any other appropriate control mechanism such as controller 93. Any of such mechanisms 92, 93, 94, 96, 100 can incorporate feedback control (illustrated by broken lines), not only of a visual nature, provided via a laparoscope 68, but also of the ultrasound imaging type via probe 90.

As shown in FIG. 9, the probe 390 includes a segmented, curved rectangular elliptical transducer 400 of the general type described in, for example, WO 99/49788. The transducer 400 has a central segment 402 which is used both for visualization and therapy and outer segment(s) 104 which is (are) used for therapy, in accordance with known principles. However, it will immediately be appreciated that other single element or multi-segment transducer configurations, such as ones providing variable focal length, can be used to advantage in other embodiments of the invention. Some of such variable focal length configurations, and driving and receiving systems for them, are described in the prior art incorporated herein by reference. Other systems are disclosed in U.S. application Ser. No. 11/070,371 and PCT Application US 2005/015648 both of which are incorporated by reference herein.

The structure of the laparoscopic probe 390 is composed of two main components, the main body or frame, and the probe tip assembly 410. The frame is illustratively constructed of aluminium plates and cylindrical pieces coupled with stainless steels rails. The aluminum plates are located near the rear of the probe. The plates hold a linear motor in place and create the space necessary for a linear screw drive to achieve the desired linear, back and forth, motion. The linear motion is translated to a hexagonal shaft which passes through a rotor enclosed by a sector motor. The sector motor controls a series of magnets bonded to the rotor which enables the rotor to rotate, creating the angular (sector) motion. The electronics are relayed through a circuit board mounted atop the stainless steel rails that support the frame plates. The main body is enclosed by a housing consisting of two shells that are currently made from a stereo lithography process. The shells are illustratively made from injection molded material such as Ultem® resin.

The frame illustratively provides a drive mechanism 92 for moving the transducer 400 back and forth in the direction of double headed arrow 406 in FIG. 2 (50 mm minimum movement), and also to rotate the transducer 400 about its axis 407 as illustrated by arrow 408. It is understood that other suitable drive mechanism(s) 92 may be used to move the transducer 400 (90° minimum rotation (+/−45°).

The probe tubing assembly 410 is primarily made from stainless steel. There are illustratively two bushings that guide the water tubing to the transducer as well as provide support for access to the coupling of the transducer shaft 409 and the hexagonal shaft. The transducer shaft 409 is coupled to the hex shaft (mentioned above) and is able to rotate and translate for both imaging and continuous HIFU Treatment.

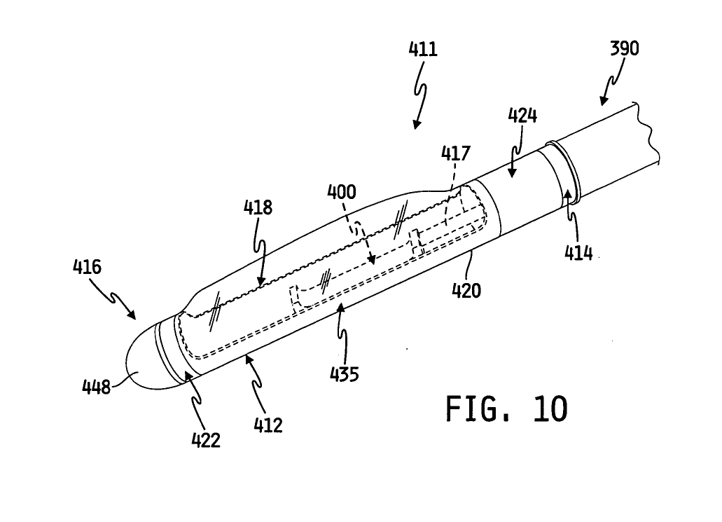

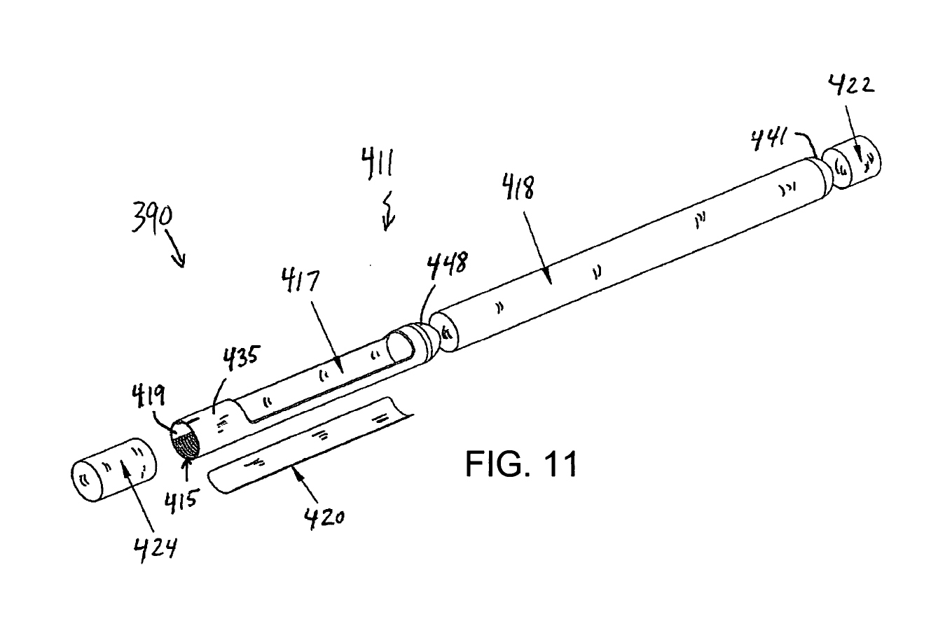

The probe tip consists of two components: a main stainless steel tubing 410 shown in FIG. 9 which has a 17 mm diameter or less to fit into an 18 mm trocar 60, and a removable tip assembly 411 shown in FIGS. 10-12. The main tubing 410 has a threaded end 413 that connects with threads formed in distal end 414 of the removable tip 411. The removable tip 411 also includes a distal end 416 having a rounded tip 148 coupled thereto. The internal threading 415 (best shown in FIG. 11) has the threads removed on opposite sides of the tubing (see area 419) to permit the transducer to pass into the tip. A coupling water bolus 418, a curved thin stainless steel shim material 420, and two short pieces of very thin heat shrink tubing 422, 424 complete the illustrated removable tip 411 components. The removable tip 411 is illustratively made from stainless steel but may be molded from a resin such as Ultem® resin or other suitable material. The bolus 418 is illustratively formed from a polyurethane membrane or condom inserted over the end of probe tip 411. Bolus 418 is illustratively a tubular membrane with a sealed end 441 best shown in FIG. 11. The shim 420 is then located over the bolus membrane 418 on an opposite side of a treatment aperture 417. Shim 420 is coupled to the tip 411 only by two heat shrinking tubes 422 and 424 best shown in FIGS. 10-12. Tubes 422 and 424 have a thickness of about 4-5 thousandths of an inch. Illustratively the membrane is made from HT-9 material available from Apex Medical. The heat shrink tubing is illustratively made from ultra thin polyester tubing and is made by Advanced Polymers.

The tubes 422 and 424 are very thin and facilitate insertion of the probe tip 411 through the trocar 60. It is understood that other securing members, such as o-rings or other suitable devices may be used to secure the bolus and the shim to the tip assembly 411. However, the tubes 422 and 424 minimize the thickness of the tip 411 which is desirable for laparoscopic procedures. Additional adhesives or other securing means are not required to secure the shim 420 to the bolus 418 or tip 411. Use of adhesives can cause weakness in the bolus membrane 418 and are therefore not desirable.

As discussed above, the removable tip 411 includes a housing 435 formed to include an opening or aperture 417. The transducer 400 is movable within the aperture as controlled by the drive mechanism 92 and controller 93 to provide the HIFU Therapy. Transducer 400 is configured to emit ultrasound energy through the aperture 417 in the direction of arrow 437 which is referred to as a treatment direction.

The housing 435, the tubes 422, 424 and the shim 420 work together to cause the bolus 418 to expand only in the treatment direction 437. The shim 420 forces the bolus 418 to expand in the direction of the opening 417 in the removable tip 411. The heat shrink tubes 422, 424 hold the shim 420 in the desired position as well as constraining the ends of the bolus membrane 418. The expansion of the water bolus 418 acoustically couples the ultrasound to the patient. It also changes the location of the transducer focus with respect to the target targeted area, thereby changing the position of the targeted tissue with respect to distance from the transducer 400.

As discussed above, the stainless steel shim 420 is an element used to control expansion of the water bolus 418 during a treatment. Removing the stainless steel shim 420 would result in a uniform expansion of the water bolus 418 around the probe tip 411 in the presence of no external objects. With no shim 420 applying pressure to hold the probe against tissue for treatment at a specific distance would result in the bolus 418 reacting by shifting water behind the probe tip and away from the tissue. This may result in a poor and uncontrolled acoustic coupling of the transducer 400 to the tissue and the inability to accurately place the HIFU Treatment zones in their desired locations.

The bolus membrane material 418 illustratively has a memory characteristic. This provides a substantially flat elevated position of bolus 418 above aperture 417 for uniform contact and coupling with a larger tissue area. Once the probe 390 is positioned within a body, a controller controls drive mechanisms to move the transducer 400 to provide HIFU Therapy.

Providing a sterile, distilled, degassed water recirculation system for cooling and acoustic coupling during treatment is another illustrated aspect of the present invention. The water should be sterile due to the required sterile surgical environment and degassed for the successful operation of the HIFU transducer.

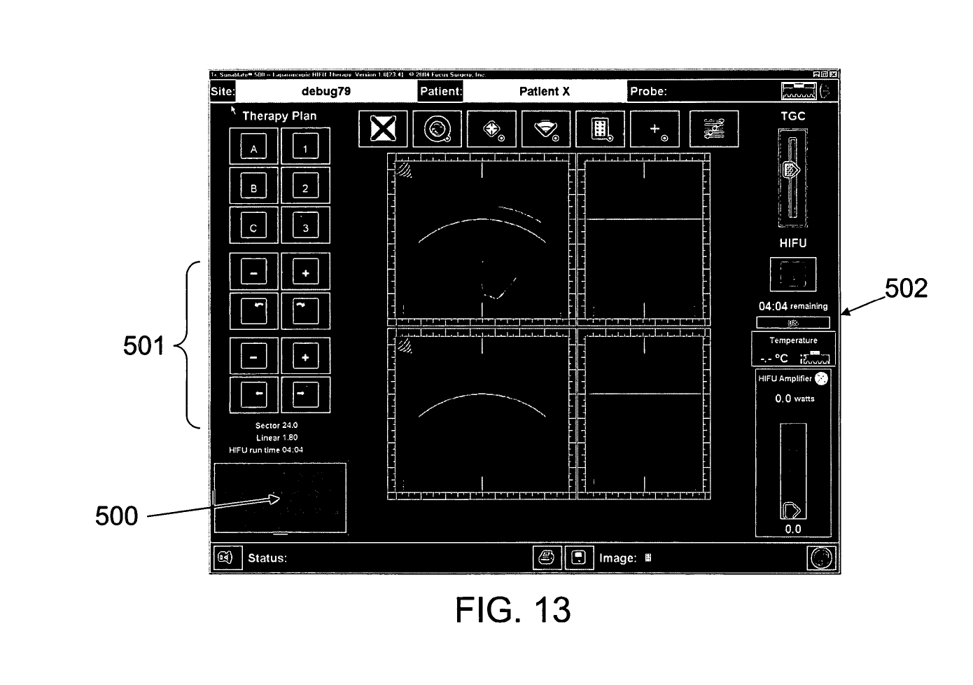

The user plans and performs the HIFU treatment using software running on the Sonablate® 500 system connected to the laparoscopic probe 390. The physician uses the real time image capability of the laparoscopic probe to aid in the final placement of the probe. When the positioning is complete, an articulated arm holding the probe 390 is locked into place. The physician judges a real time image in both sector (rotating side to side transverse to the probe axis) and linear (back and forth along probe axis) motion (“bi-plane” images). The physician then optimizes the images. Depending on the positioning and physician preference, either the linear or sector image may be chosen or the physician may alternate between the two. After physically moving the probe, fine tuning to the position of the treatment region is achieved by moving the treatment region using software controls 501 shown in FIG. 13. This adjusts the position of transducer 400 within the probe housing 435 resulting in fine tuning of the tissue treatment area. FIG. 13 displays an illustrated user interface with the treatment zones moved from the default center positions. Additional probe positioning control in depth is provided by adjusting the water volume in the coupling bolus.

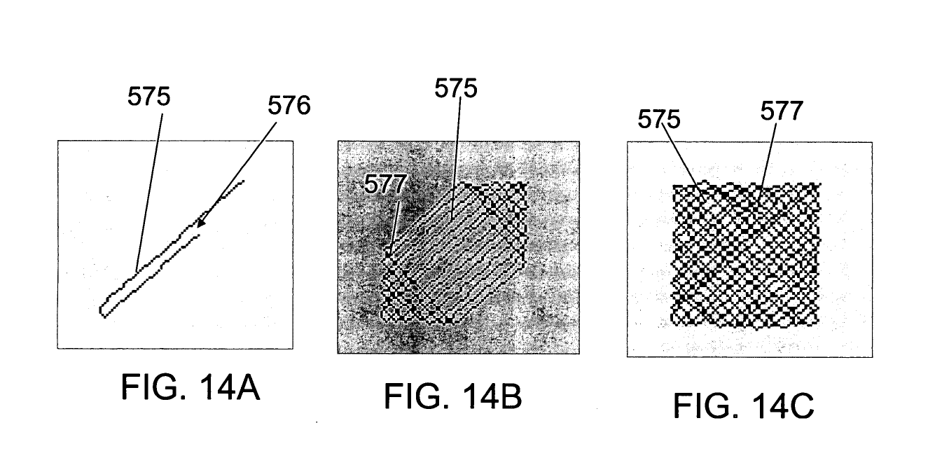

Once the treatment zone is positioned and resized by the physician to cover the desired tissue region (for example, a tumor), the HIFU Treatment is started and the probe begins to apply HIFU Therapy within the chosen region. The transducer trajectory is calculated by a series of algorithms that permit it to cover the entire treatment zone in a pattern illustrated in FIGS. 14A-14C. The trajectory is also designed to ensure constant equal trace spacing, meaning the spacing between the lines of the trajectory is substantially uniform throughout the region.

FIGS. 14A-14C illustrate an exemplary pattern of HIFU Therapy application during HIFU Treatment with the laparoscopic probe. FIG. 14A is representative of the treatment path 575 soon after the start of the treatment. FIG. 14B is representative of the treatment path 575 midway, and FIG. 14C is representative of the treatment path 575 near the end. The tracings depict the linear (vertical) and the sector (angular) positions of the transducer 400 during the treatment. This user feedback is continuously updated during the treatment.

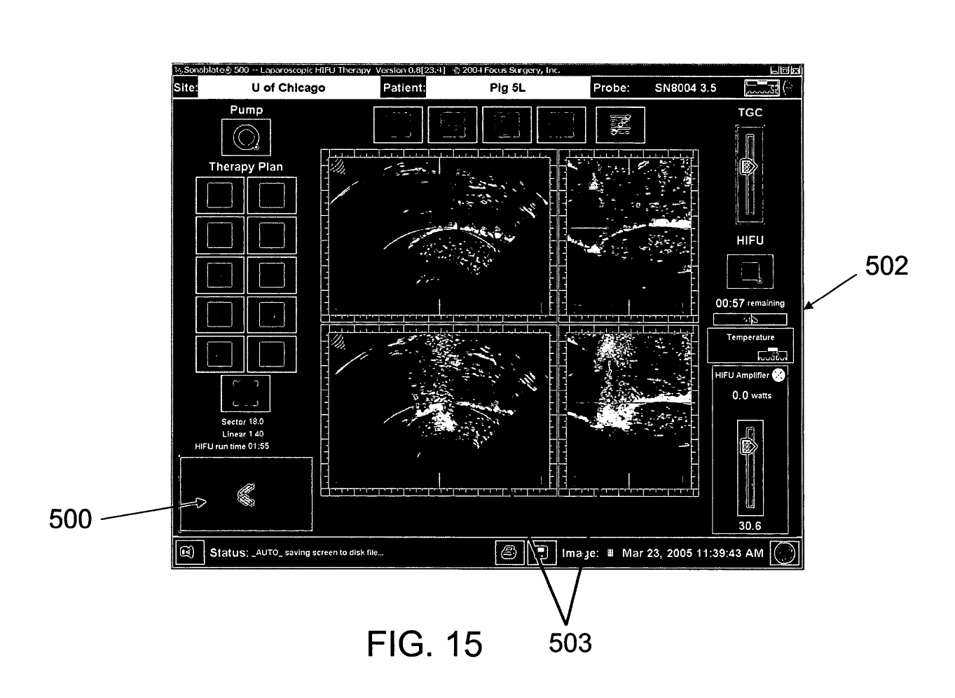

Once the treatment starts, the transducer is continuously moving at constant speed and continuously applying HIFU to the tissue treatment area. This continuous application of acoustic power is interrupted during regular intervals to image for the following reasons: 1) the images confirm that the probe has not moved with respect to the desired treatment region and 2) the images permit the user to see changes in the echogenicity (“popcorn”) of the tissue within the treatment region. This increased echogenicity (see the bottom images in FIG. 15) is an indication of the success of the application of HIFU. In other words, the system uses a “continuous on” treatment, stopping after a predetermined time interval (illustratively about every 30 seconds) for imaging. Imaging typically takes about 1 second or less. During a HIFU “continuous on” treatment, tissue ablation starts at the focal zone of the transducer. As additional HIFU energy is deposited into the tissue during the HIFU “continuous on” mode, the tissue located in the transducer pre-focal zone (located between the transducer focal zone and the transducer) is also ablated until the ablation zone extends all the way to the tissue surface (or tissue/bolus interface). This “continuous on” treatment modality has the advantage of ablating large tissue volumes in a short period of time in a controlled way (i.e. as defined by the treatment plan and treatment path), and is especially suitable for HIFU treatments in which intervening tissue is not to be spared, but ablated as well. (Compare to transrectal HIFU treatments of the prostate, in which the rectal wall/mucosa, located between the transducer and its focal zone must be spared. A “continuous on” treatment for such applications would not be prudent.) Finally, tissue surface blanching is a direct consequence of the tissue ablated region propagating all the way from the transducer focal zone to the tissue surface, and provides additional treatment feedback to the physician. Note that once the initial focal zone tissue ablation occurs, tissue properties change (absorption, impedance, attenuation), preventing the additional HIFU energy being delivered to the tissue to ablate tissue located behind the transducer focal zone. Thus, in this manner, such “continuous on” HIFU therapies are also self-limiting, as only the tissue located between the transducer face and its focal zone is ablated.

FIG. 15 illustrates an image update taken with the imaging transducer during treatment. The upper panels show the tissue before application of HIFU Treatment. The lower panels display the images acquired during the HIFU Treatment. Treatment progress may be gauged by the tracing in position 500 the lower left corner, by the time remaining 502 along the right side of the screen, and by the HIFU-induced echogenic tissue changes visible in the “during/after” image 503.

The screenshot shown in FIG. 15 was taken about half way through a HIFU Treatment. In the bottom left HIFU run time indicates that this particular treatment has lasted 1 minute and 55 seconds and the time remaining 502 (on the right side) shows 57 seconds.

The treatment algorithms of the present invention are designed to substantially fill a treatment zone or region selected by the physician. Often, these treatment zones or regions are not symmetrically shaped. Software of the present invention controls a controller 93 to move the transducer 400 back and forth in the direction of double headed arrow 406 in FIG. 9 and to rotate the transducer about its axis 407 as illustrated by arrow 408 in FIG. 9 to provide a continuous treatment path within the selected treatment region. As illustrated in FIGS. 14A, 14B and 14C, the transducer moves at constant speed, (about 1-2 mm/sec.) to provide spacing between the treatment path followed by the transducer of about 1.5-2.0 mm. The algorithm is designed to keep the spacing between adjacent portions of treatment path 575 substantially constant and to cross or intersect a previous portion of the treatment path 575 at an angle as close to 90 degrees as possible (see, for example, intersections 577 in FIGS. 14B and 14C) to avoid retracing the path 575. This pattern of path spacing at essentially 90 degree crossing provides a more uniform heat distribution with respect to depth inside the treatment region. When path 575 hits a boundary edge of a treatment zone defined by a physician, the path 575 changes directions at an angle of about 90°. In FIGS. 14A-14C, the physician defined a square treatment zone best shown by the filled zone in FIG. 14C. It is understood, however, that the treatment regions may be defined in any desired shape (typically rectangular) and are often not square.

The efficacy, performance, utility, and practicality of these newly developed Sonablate® Laparoscopic (SBL) probes and treatment methodologies was evaluated in-vivo using a pig model. Pre-selected kidney volumes (1 cm³ to 18 cm³) were targeted for ablation (including the upper and lower poles, and regions adjacent to the collective system and ureter), and treated laparoscopically with HIFU in a sterile environment using the laparoscopic probes operating in the “continuous on” mode. Integrated ultrasound image guidance was used for probe positioning, treatment planning, and treatment monitoring. The kidneys were removed either 4 or 14 days post-HIFU, and the resulting lesions were compared to the treatment plan. Results indicate that HIFU can be used laparoscopically to ablate tissue at a rate of approximately 1 to 2 cm³/minute, even in highly perfused organs like the kidney. Results also indicate that treatment methodologies vary depending on the target location, intervening tissue, probe location, and port location.

Although the invention has been described in detail with reference to certain illustrated embodiments, variations and modifications exist within the spirit and scope of the invention as described and defined in the following claims.

1-25. (canceled)

26. An apparatus for treating a targeted area of a tissue, the apparatus comprising:
a housing formed to include an aperture therein;
a transducer located within the housing, the transducer configured to emit ultrasound energy through the aperture in the housing to provide HIFU Therapy to the targeted area, the transducer configured to sense ultrasound energy;
an expandable membrane coupled to the housing, the membrane being configured to expand in a treatment direction to couple the transducer to the targeted area acoustically; and
a controller coupled to the transducer, the controller configured to cause the transducer to provide substantially continuous HIFU Therapy to a treatment path within the targeted area of tissue for a predetermined period of time, the controller switching to an imaging mode after the predetermined period of time wherein images of the tissue are obtained by ultrasound energy sensed by the transducer, the controller resuming the substantially continuous HIFU Therapy after the images are obtained.
a shim positioned over the bolus on an opposite side of the housing from the aperture, the shim being configured to block expansion of the bolus in a direction opposite from the treatment direction;
first and second coupling members located on the housing at opposite ends of the aperture and circumscribing the bolus, the first and second coupling members being configured to couple first and second portions of the bolus and first and second portions of the shim, respectively, to the housing; and
a fluid circulation system coupled to the housing to control a supply of a fluid to the housing to expand the bolus in the treatment direction prior to providing HIFU Therapy to the targeted area.

27. The apparatus of claim 26, wherein the membrane is a flexible tubular membrane inserted over the housing so that a portion of the membrane covers the aperture.

28. The apparatus of claim 27, wherein the membrane has a memory characteristic to provide a substantially flat elevated surface of the membrane located above the aperture formed in the housing.

29. The apparatus of claim 26, wherein first and second coupling members are first and second heat-shrink tubes, respectively, located on the housing at opposite ends of the aperture, the first and second heat-shrink tubes being configured to couple first and second portions of the bolus, respectively, to the housing.

30. The apparatus of claim 29, wherein the first and second heat-shrink tubes each have a thickness less than or equal to about 5/1000 of an inch.

31. The apparatus of claim 26, further comprising a drive mechanism coupled to the transducer, the drive mechanism being configured to move the transducer back and forth along a longitudinal axis of the transducer and to rotate the transducer about the longitudinal axis to provide HIFU Therapy, and a controller coupled to the drive mechanism.

32. The apparatus of claim 26, wherein the housing is configured to be inserted through a trocar for performing a laparoscopic procedure, the fluid circulation system being configured to expand the membrane after the housing, transducer, and membrane are inserted into a patient through the trocar.

33. The apparatus of claim 26, wherein a fluid circulation system provides degassed, distilled and sterile water to the housing.

34. The apparatus of claim 26, wherein the controller is configured to cause the transducer to provide substantially continuous HIFU Therapy such that a transducer focal zone tissue region is ablated.

35. The apparatus of claim 26, wherein the apparatus is configured to provide HIFU Therapy to a HIFU focal zone to modify at least one property of the tissue in the HIFU focal zone such that tissue located between the transducer and the focal zone is ablated.

36. The apparatus of claim 35, wherein the property of the tissue includes at least one of absorption, impedance and attenuation.

37. The apparatus of claim 26, wherein the predetermined period of time is substantially longer than the time required to obtain the images in the imaging mode.

38. The apparatus of claim 26, wherein the predetermined period of time is about 30 seconds.

39. The apparatus of claim 26, wherein the imaging mode has a duration of about 1 second.

40. The apparatus of claim 26, wherein the controller changes direction of the treatment path by an angle of about 90 degrees when the treatment path reaches a boundary of the treatment region defined by the physician.

41. The apparatus of claim 34, wherein at least one tissue property of the focal zone tissue region changes such that HIFU energy is not delivered to any tissue region located at a distance further from the focal zone tissue region away from the transducer.

42. The apparatus of claim 41, wherein the tissue property includes at least one of absorption, impedance, and attenuation.

43. A method for treating a targeted area of tissue, the method comprising the steps of:
administering a substantially continuous HIFU Therapy to a treatment path within the targeted area of tissue for a predetermined period of time;
stopping the HIFU Therapy treatment after the predetermined period of time;
generating a visual representation of the tissue; and
resuming the HIFU Therapy treatment after the visual representation is obtained.

44. The method of claim 43, wherein the step of administering the HIFU Therapy results in a focal zone tissue region being ablated.

45. The method of claim 43 further comprising the step of providing HIFU Therapy to a HIFU focal zone to modify at least one property of the tissue in the HIFU focal zone such that tissue located between the transducer and the focal zone is ablated.

46. The method of claim 45, wherein the property of the tissue includes at least one of absorption, impedance and attenuation.

47. The method of claim 43, wherein the predetermined period of time is substantially longer than the time required to obtain the images in the imaging mode.

48. The method of claim 43, wherein the predetermined period of time is about 30 seconds.

49. The method of claim 43, wherein the imaging mode has a duration of about 1 second.

50. The method of claim 44, wherein at least one tissue property of the focal zone tissue region changes such that HIFU energy is not delivered to any tissue region located at a distance further from the focal zone tissue region away from the transducer.

51. The method of claim 50, wherein the tissue property includes at least one of absorption, impedance, and attenuation.

52. The method of claim 43, wherein the HIFU Therapy is administered by a transducer located within a housing.

53. The method of claim 52, wherein an expandable membrane is coupled to the housing, the expandable membrane configured to expand in a treatment direction to couple the transducer to the targeted area acoustically.

54. The method of claim 53, wherein the expandable membrane has a memory characteristic to provide a substantially flat elevated surface of the membrane above the aperture formed in the housing.

55. The method of claim 43, wherein a drive mechanism is coupled to the transducer, the drive mechanism being configured to move the transducer back and forth along a longitudinal axis of the transducer and to rotate the transducer about the longitudinal axis to provide HIFU Therapy.

56. The method of claim 55, where in a controller is coupled to the drive mechanism.

57. The method of claim 52, wherein the housing is configured to be inserted through a trocar for performing a laparoscopic procedure.

58. The method of claim 53, wherein a fluid circulation system is coupled to the housing to control a supply of a fluid to the housing to expand the membrane in a treatment direction prior to providing HIFU Therapy.

59. The method of claim 56, wherein the controller changes direction of a treatment path by an angle of about 90 degrees when the treatment path reaches a boundary of a treatment region.
